(19) United States
(12) Patent Application Publication
(10) Pub. No.: US 2021/0236467 A1
(43) Pub. Date: Aug. 5, 2021

(54) DANTROLENE FORMULATIONS AND METHODS OF THEIR USE

(71) Applicant: EAGLE PHARMACEUTICALS, INC., Woodcliff Lake, NJ (US)

(72) Inventors: Charles WESCOTT, Woodcliff Lake, NJ (US); Jill COGHLAN, Duxbury, MA (US)

(21) Appl. No.: 17/052,572

(22) PCT Filed: May 21, 2019
(86) PCT No.: PCT/US2019/033260

§ 371 Date: Nov. 3, 2020

Related U.S. Application Data
(60) Provisional application No. 62/674,394, filed on May 21, 2018.

(51) Int. Cl. A61K 31/4178; A61K 9/19; A61K 47/10; A61K 9/00

(52) U.S. Cl. CPC A61K 31/4178; A61K 9/0019; A61K 47/10; A61K 9/19

(57) ABSTRACT

The disclosure is directed to liquid formulations of dantrolene, or a pharmaceutically acceptable salt thereof, and methods of their use in the treatment of disease.

### CROSS REFERENCE TO RELATED APPLICATIONS

This application claims priority to and the benefit of U.S. Provisional patent Application No. 62/674,394, filed May 21, 2018, the disclosure of which is incorporated herein by reference in its entirety.

### TECHNICAL FIELD

The disclosure is directed to liquid formulations of dantrolene, or a pharmaceutically acceptable salt thereof, and methods of their use in the treatment of disease.

### BACKGROUND

Dantrolene (1-{[5-(4-nitrophenyl)-2-furyl]methylideneamino}imidazolidine-2,4-dione), has the structure of formula (1):

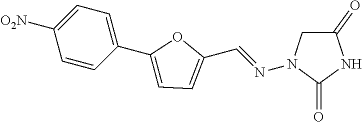 [SMILES: O=C1CN(/N=C/c2ccc(-c3ccc([N+](=O)[O-])cc3)o2)C(=O)N1]

Dantrolene is the rescue agent of choice in the treatment of malignant hyperthermia (“MH”) and is widely available in most locations where anesthetics are delivered. First synthesized in 1967, dantrolene was used initially in the treatment of muscle spasms in 1975, and later received FDA approval in 1979 for treating MH. Dantrolene is recognized as a powerful muscle relaxant and as a treatment against nerve spasticity. Since its initial discovery, dantrolene has been explored for the prophylaxis and treatment of other life-threatening conditions such as overdose from recreational drugs such as “ecstasy” (N-methyl-3,4-methylene-dioxyphenylisopropylamine), heat stroke, neuroleptic malignant syndrome, and ischemic damage to the peripheral nervous system, and may be of importance in the prevention of sudden infant death syndrome (SIDS).

Dantrolene is very poorly soluble in water. Dantrolene's poor solubility greatly impairs its administration. For example, DANTRIUM™ is dantrolene sodium supplied in 20 mg vials which must be reconstituted with 60 mL of sterile water prior to intravenous administration. The recommended dose of dantrolene for treating MH is from 1 mg/kg to about 10 mg/kg. As such, a subject weighing 80 kg would require a rapid infusion of up to 2400 mL to treat the MH.

In addition to its poor solubility, dantrolene solutions have a high pH. DANTRIUM™'s pH is about 9.5. RYANODEX®, an improved dantrolene sodium formulation that can be reconstituted to 50 mg/mL, greatly improves the speed with which dantrolene sodium can be administered. But reconstituted RYANODEX® also has a high pH—about 10.3. Because of their high pHs, currently dantrolene formulations cannot be administered subcutaneously or intramuscularly—only intravenously. Indeed, care must be taken to prevent extravasation into the surrounding tissues to avoid tissue necrosis.

There is a need for new formulations of dantrolene that are of a suitable concentration and pH, making them appropriate for intravenous administration.

### SUMMARY

The disclosure is directed to non-aqueous or anhydrous pharmaceutical compositions comprising dantrolene, or a pharmaceutically acceptable salt thereof, or a mixture thereof, and a pharmaceutically acceptable carrier comprising a C₁₋₆alkyl alcohol, a polyol, a polyether, or a mixture thereof. Methods of using these compositions to treat disorders responsive to dantrolene are also described.

### DETAILED DESCRIPTION OF ILLUSTRATIVE EMBODIMENTS

The present disclosure may be understood more readily by reference to the following detailed description taken in connection with the accompanying figures and examples, which form a part of this disclosure. It is to be understood that this disclosure is not limited to the specific compositions, devices, methods, applications, conditions, or parameters described and/or shown herein, and that the terminology used herein is for the purpose of describing particular embodiments by way of example only and is not intended to be limiting of the claimed disclosure.

As used in the specification including the appended claims, the singular forms “a,” “an,” and “the” include the plural, and reference to a particular numerical value includes at least that particular value, unless the context clearly dictates otherwise.

When a range of values is expressed, an exemplary embodiment includes from the one particular value and/or to the other particular value. All ranges are inclusive and combinable. Further, reference to values stated in ranges includes each and every value within that range. For example, the expression “from about 2 to about 4” also discloses the range “from 2 to 4.” When values are expressed as approximations, by use of the preposition “about,” it will be understood that the particular value forms another embodiment. The term “about” as used herein when referring to a measurable value such as an amount, a temporal duration, and the like, is meant to encompass reasonable variations of the value, such as, for example, ±10% from the specified value. For example, the phrase “about 50%” can include ±10% of 50, or from 45% to 55%, inclusive of 50%.

It is to be appreciated that certain features of the disclosure which are, for clarity, described herein in the context of separate embodiments, may also be provided in combination in a single embodiment. Conversely, various features of the disclosure that are, for brevity, described in the context of a single embodiment, may also be provided separately or in any subcombination.

It is to be appreciated that certain features of the disclosure which are, for clarity, described herein in the context of separate embodiments, may also be provided in combination in a single embodiment. Conversely, various features of the disclosure that are, for brevity, described in the context of a single embodiment, may also be provided separately or in any subcombination. Further, reference to values stated in ranges includes each and every value within that range.

As used herein, whether by itself or in conjunction with another term or terms, it should be understood that the phrases “method of treating” and “method of treatment” may be used interchangeably with the phrase “for use in the treatment of” a particular disease.

As used herein, whether by itself or in conjunction with another term or terms, “pharmaceutically acceptable” indicates that the designated entity such as, for example, a pharmaceutically acceptable excipient, is generally chemically and/or physically compatible with other ingredients in a composition, and/or is generally physiologically compatible with the recipient thereof.

As used herein, the term “pharmaceutical composition” shall mean a composition that is suitable for administration to humans and that contains pharmaceutically acceptable excipients, e.g., without limitation, stabilizers, bulking agents, buffers, carriers, diluents, vehicles, solubilizers, and binders.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients are enumerated in, for example, Remington's Pharmaceutical Sciences, 17^{th} ed., Mack Publishing Co. (1985).

The term “pharmaceutically acceptable salt” as used herein means a salt of a compound of the disclosure that is pharmaceutically acceptable and that possesses the desired pharmacological activity of the parent compound. In particular, such salts are non-toxic, and may be inorganic or organic acid addition salts and base addition salts. Specifically, such salts include: salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

As used herein, whether by themselves or in conjunction with another term or terms, “subject(s),” “individual(s),” and “patient(s)”, refer to mammals, including humans. The term human(s) refers to and includes, a human child, adolescent, or adult.

As used herein, whether by themselves or in conjunction with another term or terms, “treats,” “treating,” “treated,” and “treatment,” refer to and include ameliorative, palliative, and/or curative uses and results, or any combination thereof. In other embodiments, the methods described herein can be used prophylactically. It should be understood that “prophylaxis” or a prophylactic use or result do not refer to nor require absolute or total prevention (i.e., a 100% preventative or protective use or result). As used herein, prophylaxis or a prophylactic use or result refers to uses and results in which administration of a compound or composition diminishes or reduces the severity of a particular condition, symptom, disorder, or disease described herein; diminishes or reduces the likelihood of experiencing a particular condition, symptom, disorder, or disease described herein; or delays the onset or relapse (reoccurrence) of a particular condition, symptom, disorder, or disease described herein; or any combination of the foregoing.

As used herein, whether used alone or in conjunction with another term or terms, “therapeutic” and “therapeutically effective amount” refer to an amount of a compound or composition that (a) treats a particular condition, symptom, disorder, or disease described herein; (b) attenuates, ameliorates, or eliminates one or more symptoms of a particular condition, disorder, or disease described herein; (c) delays the onset or relapse (reoccurrence) of a particular condition, symptom, disorder, or disease described herein. It should be understood that the terms “therapeutic” and “therapeutically effective” encompass any one of the aforementioned effects (a)-(c), either alone or in combination with any of the others (a)-(c).

As used herein, “ameliorate” refers to the lessening of the severity in a disorder or condition being treated in a particular subject or subject population.

The disclosure is directed to liquid pharmaceutical compositions comprising dantrolene (also referred to herein as “dantrolene acid”), or a pharmaceutically acceptable salt thereof, or a mixture thereof (i.e. a mixture of dantrolene and a pharmaceutically acceptable salt of dantrolene) and a pharmaceutically acceptable carrier. The carrier is a liquid carrier comprising a C₁₋₆ alkyl alcohol, a polyol, or a mixture thereof. In the most preferred embodiments, the compositions are solutions, i.e., liquids wherein the solute(s) are dissolved in the carrier.

The disclosure is also directed to lyophilized pharmaceutical compositions comprising a mixture of dantrolene (i.e., dantrolene acid) and a pharmaceutically acceptable salt of dantrolene. Preferred ratios of dantrolene:dantrolene salt in these lyophilized pharmaceutical compositions include, for example, 90:10 to 70:30, preferably 90:10, 80:20, 75:25, or 70:30. Preferably in these aspects, the lyophilized pharmaceutical compositions comprise 90, 89, 88, 87, 86, 85, 84, 83, 82, 81, 80, 79, 78, 77, 76, 75, 74, 73, 72, 71, or 70% (w/w) of dantrolene, based on the combined weight of the dantrolene and the dantrolene salt, with the remainder being the dantrolene salt (10, 11, 12, 13, 14, 15, 16, 17, 18, 19, 20, 21, 22, 23, 24, 25, 26, 27, 28, 39, or 30% (w/w) of dantrolene salt). In these aspects, when the lyophilized pharmaceutical composition is reconstituted with a pharmaceutically acceptable carrier that is a C₁₋₆alkyl alcohol, a polyol, a polyether, or a mixture thereof, the reconstituted lyophilized pharmaceutical composition will exhibit an effective pH of 4 to about 9, for example, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, or 9.5.

In some aspects, the pharmaceutical compositions comprise dantrolene and exclude pharmaceutically acceptable salts of dantrolene. In these aspects, the pharmaceutical compositions comprise more than 95% (w/w) of dantrolene, based on the combined weight of the dantrolene and the dantrolene salt. Preferably in these aspects, the pharmaceutical compositions comprise 96, 97, 98, 99, or greater than 99% (w/w) of dantrolene, based on the combined weight of the dantrolene and the dantrolene salt.

In some aspects, the pharmaceutical compositions comprise a pharmaceutically acceptable salt of dantrolene and exclude dantrolene. In these aspects, the pharmaceutical compositions comprise more than 95% (w/w) of the dantrolene salt, based on the combined weight of the dantrolene and the dantrolene salt. Preferably in these aspects, the pharmaceutical compositions comprise 96, 97, 98, 99, or greater than 99% (w/w) of dantrolene salt, based on the combined weight of the dantrolene and the dantrolene salt.

In some aspects, the pharmaceutical compositions comprise dantrolene and a pharmaceutically acceptable salt of dantrolene. In some aspects, the pharmaceutical compositions comprise dantrolene and dantrolene sodium. In these aspects, the pharmaceutical compositions can comprise about 5, 10, 15, 20, 25, 30, 35, 40, 45, 50, 55, 60, 65, 70, 75, 80, 85, 90, or about 95% (w/w) of dantrolene, based on the combined weight of the dantrolene and the dantrolene salt. In other aspects, the pharmaceutical compositions can comprise about 5, 10, 15, 20, 25, 30, 35, 40, 45, 50, 55, 60, 65, 70, 75, 80, 85, 90, or about 95% (w/w) of dantrolene salt. Preferred ratios of dantrolene:dantrolene salt include, for example, 90:10, 80:20, 75:25, 70:30. Other ratios of dantrolene:dantrolene salt include 60:40, 50:50, 40:60, 30:70, 25:75, 20:80, and 10:90.

In those aspects wherein the pharamceutical composition is a lyophilized pharmaceutical composition comprising a mixture of dantrolene and a pharmaceutically acceptable salt of dantrolene,

A preferred dantrolene salt is dantrolene sodium. Other dantrolene salts are also within the scope of the disclosure.

The pharmaceutical compositions of the disclosure are preferably non-aqueous. As used herein, “non-aqueous” refers to compositions having 10% (w/v) or less of water. In preferred aspects, “non-aqueous” compositions have 5% (w/v) or less of water. For example, “non-aqueous” compositions can contain 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.5, 6, 6.5, 7, 7.5, 8, 8.5, 9, 9.5, or 10% (w/v) of water.

In other aspects, the pharmaceutical compositions of the disclosure are anhydrous. As used herein, “anhydrous” refers to compositions having less than 0.1% (w/v) of water. For example, “anhydrous” compositions can include an amount of water that is below the limits of detection using conventional methods and instrumentation.

The pharmaceutical compositions of the disclosure have an effective pH of 3 to 11.5. As used herein, “effective pH” refers to the pH of non-aqueous or anhydrous compositions, as measured using the methods described herein. For example, the pharmaceutical compositions of the disclosure have an effective pH of 3, 3.5, 4, 4.5, 5, 5.5, 6, 6.5, 7, 7.5, 8, 8.5, 9, 9.5, 10, 10.5, 11, or 11.5. In some aspects, the effective pH of the pharmaceutical compositions is 4 to 9. In other aspects, the effective pH of the pharmaceutical compositions is 5 to 8. In other aspects, the effective pH of the pharmaceutical compositions is about physiological pH, that is, 7.4.

The pharmaceutical compositions of the disclosure also include a pharmaceutically acceptable carrier. The carrier is a liquid carrier and comprises a C₁₋₆alkyl alcohol, a polyol, a polyether, or a mixture thereof. As used herein, a “polyol” is a liquid organic composition that includes at least two hydroxyl (—OH) moieties. As used herein, a “polyether” is a liquid organic composition that includes at least two alkenyl ether moieties.

Pharmaceutical compositions of the disclosure can have dantrolene present at a concentration of about 1 mg/mL to about 200 mg/mL, preferably 5 mg/mL to about 125 mg/mL. In particular embodiments of the disclosure, dantrolene is present at a concentration equal to or greater than about 5 mg/mL. In particular embodiments of the disclosure, dantrolene is present at a concentration equal to or greater than about 6 mg/mL. In particular embodiments of the disclosure, dantrolene is present at a concentration equal to or greater than about 7 mg/mL. In particular embodiments of the disclosure, dantrolene is present at a concentration equal to or greater than about 8 mg/mL. In particular embodiments of the disclosure, dantrolene is present at a concentration equal to or greater than about 9 mg/mL. In particular embodiments of the disclosure, dantrolene is present at a concentration equal to or greater than about 10 mg/mL. In further embodiments, dantrolene is present at a concentration of about 10 to 25 mg/mL. In still further embodiments, dantrolene is present at a concentration of about 1 mg/mL, 5 mg/mL, 10 mg/mL, 15 mg/mL, 20 mg/mL, 25 mg/mL, 30 mg/mL, 35 mg/mL, 40 mg/mL, 45 mg/mL, or 50 mg/mL. In still further embodiments, dantrolene is present at a concentration of about 125 mg/mL, 150 mg/mL, 175 mg/mL, or about 200 mg/mL.

In certain embodiments dantrolene is present at a concentration equal to or greater than about 55 mg/mL. In further embodiments, dantrolene is present at a concentration of about 55 to 125 mg/mL. In particular embodiments, is present at a concentration of about 75 mg/mL, 80 mg/mL, 85 mg/mL, 90 mg/mL, 95 mg/mL, 100 mg/mL, 105 mg/mL, 110 mg/mL, 115 mg/mL, 120 mg/mL or 125 mg/mL. In other embodiments, is present at a concentration of about 75 mg/mL to 95 mg/mL, 80 mg/mL to 100 mg/mL, 90 mg/mL to 110 mg/ml, 95 mg/mL to 105 mg/mL, 95 mg/mL to 115 mg/mL, 100 mg/mL to 110 mg/mL, 110 mg/mL to 125 mg/mL, including all ranges and subranges there between.

According to the disclosure, the C₁₋₆alkyl alcohol is an alcohol suitable for administration to humans. A preferred C₁₋₆alkyl alcohol is ethanol. The carrier can be comprised of the C₁₋₆alkyl alcohol (or a mixture of C₁₋₆alkyl alcohols) and exclude polyols. In these aspects, the carrier comprises more than 95% (v/v) of the C₁₋₆alkyl alcohol (or a mixture of C₁₋₆alkyl alcohols), for example, 96, 97, 98, 99, or greater than 99% (v/v) of the C₁₋₆alkyl alcohol (or a mixture of C₁₋₆alkyl alcohols). In other aspects, the carrier can comprise the C₁₋₆alkyl alcohol (or a mixture of C₁₋₆alkyl alcohols) in combination with one or more polyols. In these aspects, the carrier can comprise 5, 10, 15, 20, 25, 30, 35, 40, 45, 50, 55, 60, 65, 70, 75, 80, 85, 90, or 95% (v/v) of the C₁₋₆ alkyl alcohol (or a mixture of C₁₋₆alkyl alcohols), with the remainder comprising polyol(s).

According to the disclosure, the polyol is suitable for administration to humans and is an alkylene glycol (preferably a C₁₋₆alkyl glycol) or a mixture of alkylene glycols. A preferred alkylene glycol is propylene glycol. Ethylene glycol is also within the scope of the disclosure.

According to the disclosure, the polyether is suitable for administration to humans and is a liquid polyalkylether or mixture of liquid polyalkylethers. A preferred polyalkylene ether is a liquid polyethylene glycol (PEG), for example, PEG200, PEG300, PEG400, PEG500, or PEG600. PEG400 is a particularly preferred PEG. In other aspects, the polyether is a liquid “capped” polyalkylene glycol, that is, a polyalkylene glycol that is terminated on one or both termini with a non-hydroxyl moiety. Capped PEGs that are within the scope of the disclosure include polyalkylene glycol monomethyl ethers and polyalkylene glycol dimethyl ethers.

The carrier can be comprised of one polyol or one polyether, that is, one alkylene glycol, one liquid polyalkylene glycol or one liquid capped polyalkylene glycol. Preferably, the carrier is comprises of one alkylene glycol or one liquid polyalkylene glycol. In other aspects, the carrier comprises a capped polyalkylene glycol.

In other aspects, the carrier can be comprised of a polyol and a polyether, for example, a mixture of alkylene glycols, a mixture of polyalkyene glycols, a mixture of capped polyalkylene glycols, a mixture of alkylene glycol(s) and polyalkylene glycol(s), a mixture of alkylene glycol(s) and capped polyalkylene glycol(s), or a mixture of polyalkylene glycol(s) and capped polyalkylene glycol(s). In yet other aspects, the carrier can comprise a C₁₋₆alkyl alcohol, an alkyl glycol, and a liquid polyalkylene glycol. In yet other aspects, the carrier can comprise a C₁₋₆alkyl alcohol, an alkyl glycol, and a capped liquid polyalkylene glycol.

In those aspects wherein the carrier includes a polyol(s), the carrier can comprise 5, 10, 15, 20, 25, 30, 35, 40, 45, 50, 55, 60, 65, 70, 75, 80, 85, 90, or 95% (v/v) of the polyol or more than one polyol. In other aspects, the carrier can comprise more than 95% (v/v) of the polyol or more than one polyol, for example, 96, 97, 98, 99 or greater than 99% (v/v) of the polyol or more than one polyol.

In those aspects wherein the carrier includes one alkylene glycol or more than one alkylene glycol, the carrier can comprise 5, 10, 15, 20, 25, 30, 35, 40, 45, 50, 55, 60, 65, 70, 75, 80, 85, 90, or 95% (v/v) of the alkylene glycol or more than one alkylene glycol. In other aspects, the carrier can comprise more than 95% (v/v) of the alkylene glycol or more than one alkylene glycol, for example, 96, 97, 98, 99 or greater than 99% (v/v) of the alkylene glycol or more than one alkylene glycol. For example, in preferred aspects, the carrier includes propylene glycol. In these aspects, the carrier can comprise 5, 10, 15, 20, 25, 30, 35, 40, 45, 50, 55, 60, 65, 70, 75, 80, 85, 90, or 95% (v/v) of propylene glycol. In other aspects, the carrier comprises more that 95% (v/v) of propylene glycol, for example, 96, 97, 98, 99, or greater than 99% propylene glycol. In some aspects, the carrier comprises 100% of propylene glycol.

In those aspects wherein the carrier includes one polyalkylene glycol or more than one polyalkylene glycol, the carrier can comprise 5, 10, 15, 20, 25, 30, 35, 40, 45, 50, 55, 60, 65, 70, 75, 80, 85, 90, or 95% (v/v) of the polyalkylene glycol or more than one polyalkylene glycol. In other aspects, the carrier can comprise more than 95% (v/v) of the polyalkylene glycol or more than one polyalkylene glycol, for example, 96, 97, 98, 99 or greater than 99% (v/v) of the polyalkylene glycol or more than one polyalkylene glycol. For example, in preferred aspects, the carrier includes a liquid polyethylene glycol, for example PEG200, PEG300, PEG400, PEG500, or PEG600, or a combination thereof. In some aspects, the carrier can comprise 5, 10, 15, 20, 25, 30, 35, 40, 45, 50, 55, 60, 65, 70, 75, 80, 85, 90, or 95% (v/v) of the PEG. In some aspects, the carrier can comprise 5, 10, 15, 20, 25, 30, 35, 40, 45, 50, 55, 60, 65, 70, 75, 80, 85, 90, or 95% (v/v) of the mixture of PEGs. In other aspects, the carrier comprises more that 95% (v/v) of the PEG or mixture of PEGs, for example, 96, 97, 98, 99, or greater than 99% (v/v) of the PEG or 96, 97, 98, 99, or greater than 99% (v/v) of the mixture of PEGs. In some aspects, the carrier comprises 100% of a PEG, for example, 100% of PEG 200, PEG300, PEG400, PEG500, or PEG600.

In those aspects wherein the carrier includes one capped polyalkylene glycol or more than one capped polyalkylene glycol, the carrier can comprise 5, 10, 15, 20, 25, 30, 35, 40, 45, 50, 55, 60, 65, 70, 75, 80, 85, 90, or 95% (v/v) of the capped polyalkylene glycol or more than one capped polyalkylene glycol. In other aspects, the carrier can comprise more than 95% (v/v) of the capped polyalkylene glycol or more than one capped polyalkylene glycol, for example, 96, 97, 98, 99 or greater than 99% (v/v) of the capped polyalkylene glycol or more than one capped polyalkylene glycol. In some aspects, the carrier can comprise 5, 10, 15, 20, 25, 30, 35, 40, 45, 50, 55, 60, 65, 70, 75, 80, 85, 90, or 95% (v/v) of a capped polyalkylene glycol. In some aspects, the carrier can comprise 5, 10, 15, 20, 25, 30, 35, 40, 45, 50, 55, 60, 65, 70, 75, 80, 85, 90, or 95% (v/v) of the mixture of capped polyalkylene glycols. In other aspects, the carrier comprises more that 95% (v/v) of the capped polyalkylene glycol or mixture of capped polyalkylene glycols, for example, 96, 97, 98, 99, or greater than 99% (v/v) of the capped polyalkylene glycol or 96, 97, 98, 99, or greater than 99% (v/v) of the mixture of capped polyalkylene glycols. In some aspects, the carrier comprises 100% of a capped polyalkylene glycol.

In some aspects, the carrier includes ethanol, propylene glycol, and a PEG. In these aspects, the carrier can include 5, 10, 15, 20, 25, 30, 35, 40, 45, 50, 55, 60, 65, 70, 75, 80, 85, 90, or 95% (v/v) of ethanol, with the remainder comprising propylene glycol and the PEG. In other of these aspects, the carrier can include 5, 10, 15, 20, 25, 30, 35, 40, 45, 50, 55, 60, 65, 70, 75, 80, 85, 90, or 95% (v/v) of propylene glycol, with the remainder comprising ethanol and the PEG. In other of these aspects, the carrier can include 5, 10, 15, 20, 25, 30, 35, 40, 45, 50, 55, 60, 65, 70, 75, 80, 85, 90, or 95% (v/v) of the PEG, with the remainder comprising ethanol and propylene glycol.

In some aspects, the carrier includes ethanol, propylene glycol, and a capped PEG. In these aspects, the carrier can include 5, 10, 15, 20, 25, 30, 35, 40, 45, 50, 55, 60, 65, 70, 75, 80, 85, 90, or 95% (v/v) of ethanol, with the remainder comprising propylene glycol and the capped PEG. In other of these aspects, the carrier can include 5, 10, 15, 20, 25, 30, 35, 40, 45, 50, 55, 60, 65, 70, 75, 80, 85, 90, or 95% (v/v) of propylene glycol, with the remainder comprising ethanol and the capped PEG. In other of these aspects, the carrier can include 5, 10, 15, 20, 25, 30, 35, 40, 45, 50, 55, 60, 65, 70, 75, 80, 85, 90, or 95% (v/v) of the capped PEG, with the remainder comprising ethanol and propylene glycol.

In some aspects, the carrier includes ethanol and propylene glycol. In these aspects, the carrier can include 5, 10, 15, 20, 25, 30, 35, 40, 45, 50, 55, 60, 65, 70, 75, 80, 85, 90, or 95% (v/v) of ethanol, with the remainder comprising propylene glycol. In other of these aspects, the carrier can include 5, 10, 15, 20, 25, 30, 35, 40, 45, 50, 55, 60, 65, 70, 75, 80, 85, 90, or 95% (v/v) of propylene glycol, with the remainder comprising ethanol.

In some aspects, the carrier includes ethanol and a PEG. In these aspects, the carrier can include 5, 10, 15, 20, 25, 30, 35, 40, 45, 50, 55, 60, 65, 70, 75, 80, 85, 90, or 95% (v/v) of ethanol, with the remainder comprising the PEG. In other of these aspects, the carrier can include 5, 10, 15, 20, 25, 30, 35, 40, 45, 50, 55, 60, 65, 70, 75, 80, 85, 90, or 95% (v/v) of PEG, with the remainder comprising ethanol.

In some aspects, the carrier includes ethanol and a capped PEG. In these aspects, the carrier can include 5, 10, 15, 20, 25, 30, 35, 40, 45, 50, 55, 60, 65, 70, 75, 80, 85, 90, or 95% (v/v) of ethanol, with the remainder comprising the capped PEG. In other of these aspects, the carrier can include 5, 10, 15, 20, 25, 30, 35, 40, 45, 50, 55, 60, 65, 70, 75, 80, 85, 90, or 95% (v/v) of capped PEG, with the remainder comprising ethanol.

In some aspects, the carrier includes propylene glycol and a PEG. In these aspects, the carrier can include 5, 10, 15, 20, 25, 30, 35, 40, 45, 50, 55, 60, 65, 70, 75, 80, 85, 90, or 95% (v/v) of propylene glycol, with the remainder comprising the PEG. In other of these aspects, the carrier can include 5, 10, 15, 20, 25, 30, 35, 40, 45, 50, 55, 60, 65, 70, 75, 80, 85, 90, or 95% (v/v) of PEG, with the remainder comprising propylene glycol.

In some aspects, the carrier includes propylene glycol and a capped PEG. In these aspects, the carrier can include 5, 10, 15, 20, 25, 30, 35, 40, 45, 50, 55, 60, 65, 70, 75, 80, 85, 90, or 95% (v/v) of propylene glycol, with the remainder comprising the capped PEG. In other of these aspects, the carrier can include 5, 10, 15, 20, 25, 30, 35, 40, 45, 50, 55, 60, 65, 70, 75, 80, 85, 90, or 95% (v/v) of capped PEG, with the remainder comprising propylene glycol.

In certain embodiments, pharmaceutical compositions of the disclosure may further comprise an additional pharmaceutically acceptable excipient. For example, the pharmaceutical compositions of the disclosure may further include a stabilizer or two or more stabilizers. In still further embodiments of the disclosure, the stabilizer is selected from the group consisting of surfactants, polymers, cross-linked polymers, buffering agents, electrolytes, and non-electrolytes. In yet further embodiments of the disclosure, the composition comprises a combination of two or more stabilizers selected from the group consisting of surfactants, polymers, cross-linked polymers, buffering agents, electrolytes, and non-electrolytes. In yet further embodiments of the disclosure, the stabilizer is a surfactant such as, but not limited to, polysorbate 80, polysorbate 20, poloxamer 188, polyethoxylated vegetable oils, lecithin, human serum albumin, and mixtures thereof. In particular embodiments of the disclosure, the stabilizer is a polymer, such as, but not limited to, a polyvinylpyrrolidone (such as, but not limited to povidone K12, povidone K17, and mixtures thereof), polyethylene glycol 3350, and mixtures thereof. In other embodiments of the disclosure, the stabilizer is an electrolyte such as, but not limited to, sodium chloride, calcium chloride, and mixtures thereof. In still other embodiments of the disclosure, the stabilizer is a non-electrolyte, such as, but not limited to, dextrose, glycerol, mannitol, benzyl benzoate, or mixtures thereof. In other embodiments of the disclosure, the stabilizer is a cross-linked polymer such as, but not limited to, carboxymethylcellulose sodium (CMC). In some embodiments of the disclosure, the stabilizer is CMC 7LF, CMC 7MF, CMC 7HF, or mixtures thereof.

In further embodiments of the disclosure, combinations of non-electrolyte stabilizers and electrolyte stabilizers may be used. In some embodiments, the combination of stabilizers may comprise two or more non-electrolyte stabilizers. In other embodiments, the combination of stabilizers may comprise two or more electrolyte stabilizers. In further embodiments, the combination of stabilizers may comprise one or more non-electrolyte stabilizers and one or more electrolyte stabilizers. In yet further embodiments, the combination of stabilizers may comprise two or more of mannitol, dextrose, and sodium chloride.

In certain embodiments of the disclosure, combinations of surfactant stabilizers and polymer stabilizers may be used. In some embodiments, the combination of stabilizers may comprise two or more surfactant stabilizers. In other embodiments, the combination of stabilizers may comprise two or more polymer stabilizers. In further embodiments, the combination of stabilizers may comprise one or more surfactant stabilizers and one or more polymer stabilizers. In yet further embodiments, the combination of stabilizers may comprise two or more of polysorbate 80, polysorbate 20, and poloxamer 188. In still further embodiments, the combination of stabilizers may comprise one or more of polysorbate 80, polysorbate 20, and poloxamer 188 and one or more of povidone K12, povidone K17, and polyethylene glycol 3350.

In certain embodiments of the disclosure, the composition comprises about 0.2 mg/mL to about 75 mg/mL of the one or more excipients, and all ranges and subranges therebetween. In particular embodiments of the disclosure, the composition comprises about 0.2 to 0.7 mg/mL, 0.5 to 1 mg/mL, 1 to 5 mg/mL, 2 to 8 mg/mL, 5 to 6 mg/mL, 5 to 10 mg/mL, 8 to 12 mg/mL, 10 to 15 mg/mL, 15 to 20 mg/mL, 20 to 30 mg/mL, 30 to 40 mg/mL, 40 to 50 mg/mL, 45 to 55 mg/mL, 50 to 60 mg/mL, or 60 to 75 mg/mL of one or more excipients, and all ranges and subranges there between. In further embodiments of the disclosure, the composition comprises about 0.2 mg/mL, 0.5 mg/mL, 1 mg/mL, 2 mg/mL, 3 mg/mL, 4 mg/mL, 5 mg/mL, 5.5 mg/mL, 6 mg/mL, 7 mg/mL, 8 mg/mL, 9 mg/mL, 10 mg/mL, 12 mg/mL, 15 mg/mL, 17 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, or 75 mg/mL of one or more excipients.

Pharmaceutical compositions of the disclosure can be administered intravenously. Intravenously administered pharmaceutical compositions will be sterile. Alternatively, pharmaceutical compositions of the disclosure can be administered intramuscularly. Intramuscularly administered pharmaceutical compositions will be sterile. In other embodiments, pharmaceutical compositions of the disclosure are administered subcutaneously. Subcutaneously administered pharmaceutical compositions will be sterile Pharmaceutical compositions of the disclosure can also be administered orally. Orally administered pharmaceutical compositions can be sterile, however, sterility is not required. In other embodiments, pharmaceutical compositions of the disclosure are administered transmuscosally, for example via intranasal administration. Transmucosally administered pharmaceutical compositions will be sterile. In other embodiments, pharmaceutical compositions of the disclosure are administered intraosseously. Intraosseously administered pharmaceutical compositions will be sterile.

The pharmaceutical compositions of the disclosure can be administered “as-is,” that is, without the addition of any further diluents or other excipients. In other embodiments, the pharmaceutical compositions of the disclosure are diluted with a pharmaceutically acceptable diluent prior to administration.

Some aspects of the disclosure are directed to non-aqueous or anhydrous pharmaceutical composition comprising dantrolene, or a pharmaceutically acceptable salt thereof, or a mixture thereof, and a pharmaceutically acceptable carrier comprising a C₁₋₆alkyl alcohol, a polyol, a polyether, or a mixture thereof. Such carriers are described in more detail elsewhere, herein. These pharmaceutical compositions exhibit an effective pH that is physiologically compatible with administration to a human. For example, the pharmaceutical compositions exhibit an effective pH of between 4 and 9. The pharmaceutical compositions can be administered, “as is” or they can be diluted with an additional pharmaceutically acceptable carrier, for example, an aqueous carrier such as Water for Injection, Sodium Chloride Injection, Dextrose Injection, Ringer's Injection, and the like, before being administered, in a therapeutically effective amount, to the subject using any of the routes of administration described herein.

In some aspects using a lyophilized pharmaceutical composition comprising dantrolene and a pharmaceutically acceptable salt of dantrolene (prefereably dantrolene and dantrolene sodium), the lyophilized pharmaceutical composition is reconsituted with a pharmaceutically acceptable carrier that is a C₁₋₆alkyl alcohol, a polyol, a polyether, or a mixture thereof. Such carriers are described in more detail elsewhere, herein. These reconstituted lyophilized pharmaceutical compositions exhibit an effective pH that is physiologically compatible with administration to a human. For example, the reconstituted lyophilized pharmaceutical compositions exhibit an effective pH of between 4 and 9. The reconstituted lyophilized pharmaceutical compositions can be administered, “as is” or they can be diluted with an additional pharmaceutically acceptable carrier, for example, an aqueous carrier such as Water for Injection, Sodium Chloride Injection, Dextrose Injection, Ringer's Injection, and the like, before being administered, in a therapeutically effective amount, to the subject using any of the routes of administration described herein.

Pharmaceutical compositions of the disclosure can be used to treat disorders responsive to dantrolene. See, e.g., U.S. Pat. Nos. 7,758,890, 8,110,225, 8,685,460, 9,271,964, 9,603,840, 9,789,090, 9,884,044, and U.S. Provisional Application No. 62/554,049, filed Sep. 5, 2017, the entireties of which are incorporated by reference herein. In other aspects, subjects in need of treatment can be administered a therapeutically effective amount of a pharmaceutical composition of the disclosure.

Disorders responsive to dantrolene include, for example, malignant hyperthermia, chronic spasticity, exertional heat stroke, cardiac arrhythmias, tachycardis, atrial fibrillation, cardiac arrest, myocardial infarction, heart failure, myocardial injury, cardiomyopathy, central core disease, amyotrophic lateral sclerosis, rhabdomyolysis, Duchenne muscular dystrophy, ataxia, detrusor overactivity, overactive bladder, seizure, epilepsy, neuroleptic malignant syndrome, human stress disorder, Alzheimer's disease, Huntington's disease, multiple sclerosis, amyotrophic lateral sclerosis, Parkinson's disease, ischemia-reperfusion injury, neuronal reperfusion injury, hypoxia, cerebral aneurysm, subarachnoid hemorrhage, stroke, hyperthermia associated with drug abuse, or hyperthermia associated with drug overdose.

In preferred aspects, the pharmaceutical compositions of the disclosure are used to treat malignant hyperthermia in a subject.

In other aspects, the pharmaceutical compositions of the disclosure are used to treat chronic spasticity in a subject.

In other aspects, the pharmaceutical compositions of the disclosure are used to treat exertional heat stroke in a subject.

In other aspects, the pharmaceutical compositions of the disclosure are used to treat cardiac arrhythmias in a subject.

In other aspects, the pharmaceutical compositions of the disclosure are used to treat tachycardis in a subject.

In other aspects, the pharmaceutical compositions of the disclosure are used to treat atrial fibrillation in a subject.

In other aspects, the pharmaceutical compositions of the disclosure are used to treat cardiac arrest in a subject.

In other aspects, the pharmaceutical compositions of the disclosure are used to treat myocardial infarction in a subject.

In other aspects, the pharmaceutical compositions of the disclosure are used to treat heart failure in a subject.

In other aspects, the pharmaceutical compositions of the disclosure are used to treat myocardial injury in a subject.

In other aspects, the pharmaceutical compositions of the disclosure are used to treat cardiomyopathy in a subject.

In other aspects, the pharmaceutical compositions of the disclosure are used to treat central core disease in a subject.

In other aspects, the pharmaceutical compositions of the disclosure are used to treat amyotrophic lateral sclerosis in a subject.

In other aspects, the pharmaceutical compositions of the disclosure are used to treat rhabdomyolysis in a subject.

In other aspects, the pharmaceutical compositions of the disclosure are used to treat Duchenne muscular dystrophy in a subject.

In other aspects, the pharmaceutical compositions of the disclosure are used to treat ataxia in a subject.

In other aspects, the pharmaceutical compositions of the disclosure are used to treat detrusor overactivity in a subject.

In other aspects, the pharmaceutical compositions of the disclosure are used to treat overactive bladder in a subject.

In other aspects, the pharmaceutical compositions of the disclosure are used to treat seizure in a subject.

In other aspects, the pharmaceutical compositions of the disclosure are used to treat epilepsy in a subject.

In other aspects, the pharmaceutical compositions of the disclosure are used to treat neuroleptic malignant syndrome in a subject.

In other aspects, the pharmaceutical compositions of the disclosure are used to treat human stress disorder in a subject.

In other aspects, the pharmaceutical compositions of the disclosure are used to treat Alzheimer's disease in a subject.

In other aspects, the pharmaceutical compositions of the disclosure are used to treat Huntington's disease in a subject.

In other aspects, the pharmaceutical compositions of the disclosure are used to treat multiple sclerosis in a subject.

In other aspects, the pharmaceutical compositions of the disclosure are used to treat Parkinson's disease in a subject.

In other aspects, the pharmaceutical compositions of the disclosure are used to treat ischemia-reperfusion injury in a subject.

In other aspects, the pharmaceutical compositions of the disclosure are used to treat neuronal reperfusion injury in a subject.

In other aspects, the pharmaceutical compositions of the disclosure are used to treat hypoxia in a subject.

In other aspects, the pharmaceutical compositions of the disclosure are used to treat cerebral aneurysm in a subject.

In other aspects, the pharmaceutical compositions of the disclosure are used to treat subarachnoid hemorrhage in a subject.

In other aspects, the pharmaceutical compositions of the disclosure are used to treat stroke in a subject.

In other aspects, the pharmaceutical compositions of the disclosure are used to treat hyperthermia associated with drug abuse (e.g., ecstasy (3,4-Methylenedioxymethamphetamine) abuse) in a subject.

In other aspects, the pharmaceutical compositions of the disclosure are used to treat hyperthermia associated with drug overdose (e.g., ecstasy (3,4-Methylenedioxymethamphetamine) overdose) in a subject.

In other aspects, the pharmaceutical compositions of the disclosure are used to treat acetylcholine accumulation in a subject.

In other aspects, the compounds and/or pharmaceutical compositions of the disclosure are used to treat neurotoxic nerve agent exposure, for example, e.g., organophophorus agents such as sarin, soman, and VX in a subject. As used herein, “neurotoxic nerve agent” or “nerve agent” refers to compounds that affect the transmission of nerve impulses in the nervous system. Nerve agents are organophosphorus compounds, that is, they are of the formula (R)₃P(O), wherein each R group can be the same or different. “G”-type nerve agents include O-pinacolyl methylphosphonofluoridate (soman, GD), ethyl N,N-dimethylphosphoramidocyanidate (tabun, GA), propan-2-yl methylphosphonofluoridate (sarin, GB), cyclohexyl methylphosphonofluoridate (cyclosarin, GF), and 2-(Dimethylamino)ethyl (GV). “V”-type nerve agents include O-cyclopentyl S-(2-diethylaminoethyl) methylphosphonothiolate (EA-3148), (S)-(ethyl {[2-(diethylamino)ethyl]sulfonyls}(ethyl)phosphonates) such as (S)-(ethyl {[2-(diethylamino)ethyl]sulfanyl}(ethyl)phosphinate) (VE), 0,0-Diethyl S-[2-(diethylamino)ethyl]phosphorothioate (VG), S[2-(Diethylamino)ethyl]O-ethyl methylphosphonothioate (VM), N,N-diethyl-2-(methyl-(2-methylpropoxy)phosphoryl)sulfanylethanamine (VR), and Ethyl ({2-[bis(propan-2-yl)amino]ethyl}sulfanyl)(methyl)phosphinate (VX). The methods described herein can be used to treat a subject exposed to one nerve agent. The methods described herein can also be used to treat a subject exposed to two or more nerve agents.

The amount of dantrolene, or a pharmaceutically acceptable salt thereof, that is therapeutically effective to treat the subject according to any of the described methods should be determined by a practitioner skilled in the art. In those embodiments wherein the subject is human, the therapeutically effective amount of the dantrolene is 1 mg/kg to about 30 mg/kg, which may be administered in one dose or more than one dose. In other aspects, the therapeutically effective amount of dantrolene is 1 mg/kg to about 20 mg/kg. In other aspects, the therapeutically effective amount of dantrolene is about 5 mg/kg to about 30 mg/kg. In other aspects, the therapeutically effective amount of dantrolene is about 10 mg/kg to about 30 mg/kg. In other aspects, the therapeutically effective amount of dantrolene is about 15 mg/kg to about 30 mg/kg. In other aspects, the therapeutically effective amount of dantrolene is about 20 mg/kg to about 30 mg/kg. In other aspects, the therapeutically effective amount of dantrolene is about 5 mg/kg to about 20 mg/kg. In other aspects, the therapeutically effective amount of dantrolene is about 5 mg/kg to about 15 mg/kg. In other aspects, the therapeutically effective amount of dantrolene is about 5 mg/kg to about 10 mg/kg. In other aspects, the therapeutically effective amount of dantrolene is about 10 mg/kg to about 20 mg/kg. In other aspects, the therapeutically effective amount of dantrolene is about 2 mg/kg to about 10 mg/kg, preferably from about 2 mg/kg to about 6 mg/kg. In other aspects, the therapeutically effective amount of dantrolene is about 15 mg/kg to about 20 mg/kg. In other aspects, the therapeutically effective amount of dantrolene is about 1, 2, 3, 4, 5, 6, 7, 8, 9, 10, 11, 12, 13, 14, 15, 16, 17, 18, 19, 20, 21, 22, 23, 24, 25, 26, 27, 28, 29, or about 30 mg/kg. In some embodiments, the therapeutically effective amount of dantrolene for treating a human subject is greater than 30 mg/kg, for example, 30 mg/kg to about 100 mg/kg, which can be administered in one or two doses. In some aspects, the therapeutically effective amount of dantrolene for treating a human subject is about 35, 40, 45, 50, 55, 60, 65, 70, 75, 80, 85, 90, 95, or about 100 mg/kg.

The following examples are provided to illustrate some of the concepts described within this disclosure. While each example is considered to provide specific individual embodiments of disclosure, none of the Examples should be considered to limit the more general embodiments described herein. In the following examples, efforts have been made to ensure accuracy with respect to numbers used (e.g. amounts, temperature, etc.) but some experimental error and deviation should be accounted for.

### EXAMPLES

### Example 1. Solubility and pH in PEG400

11.2 mgs of dantrolene acid was added to 224 uL of PEG400. The liquid was cloudy after vortexing and some solid remained at the bottom of the Eppendorf tube, indicating that a saturated solution had been achieved. The tube was centrifuged for 5 min at 15,000 rpm at room temperature to separate the sample into supernatant (dantrolene acid in solution) and pellet (insoluble material). The supernatant was transferred into a clean tube and diluted 100-, 200- and 400-fold in 25% acetonitrile for analysis by UPLC as described below. The solubility of dantrolene acid in PEG400 was calculated as −15 mg/mL.

The pH of the supernatant was measured by making a 10-fold dilution of the supernatant in water. The pH was measured as 4.88. The 10-fold dilution precipitated out quickly.

### Example 2. Solubility and pH in Propylene Glycol

16.3 mgs of dantrolene acid was added to 326 uL of propylene glycol. The liquid was cloudy after vortexing and some solid remained at the bottom of the Eppendorf tube, indicating that a saturated solution had been achieved. The tube was centrifuged for 5 min at 15,000 rpm at room temperature to separate the sample into supernatant (dantrolene acid in solution) and pellet (insoluble material). The supernatant was transferred into a clean tube and diluted 50-, 100- and 200-fold in 25% acetonitrile for analysis by UPLC as described below. The solubility of dantrolene acid in propylene glycol was calculated as ˜0.8 mg/mL.

The effective pH of the supernatant was measured by making a 10-fold dilution of the supernatant in water. The pH was measured as 5.73. The 10-fold dilution precipitated out slowly.

### Example 3. Varying Ratios to Determine Formulation at Physiologic pH

pH in PEG400 of Varying 10 mg/mL Dantrolene Acid:Dantrolene Sodium Samples.

13.0 mgs of dantrolene acid was dissolved in 1.3 mL of PEG400, targeting 10 mg/mL dantrolene acid. 12.7 mgs of dantrolene sodium was dissolved in 1.27 mL of PEG400, targeting 10 mg/mL of dantrolene sodium. The liquid in the dantrolene acid sample was cloudy after vortexing and some solid remained at the bottom of the Eppendorf tube, indicating that a saturated solution had been achieved. The dantrolene sodium sample was not cloudy, indicating the dantrolene sodium had completely dissolved. Both tubes were centrifuged for 5 min at 3,900 rpm at room temperature to separate the sample into supernatant (dantrolene acid or dantrolene sodium in solution) and pellet (insoluble material). The dantrolene acid sample had a pellet form while the dantrolene sodium sample did not. The supernatant of the dantrolene acid sample was transferred into a clean tube. The dantrolene acid supernatant and the dantrolene sodium solution were diluted 100- and 200-fold in 25% acetonitrile for analysis on the UPLC as described below.

The effective pH of the dantrolene acid supernatant and dantrolene sodium sample was measured by making 10-fold dilutions in water, per the methods described elsewhere herein. The pH for the dantrolene acid supernatant was measured as 4.95. The pH for the dantrolene sodium was measured as 9.67.

The dantrolene acid supernatant and dantrolene sodium solution were mixed in varying ratios of acid:base (sodium) based on volume. Four separate samples at different ratios were made—

90:10 (180 uL of dantrolene acid+20 uL of dantrolene sodium)

80:20 (160 uL of dantrolene acid+40 uL of dantrolene sodium)

75:25 (150 uL of dantrolene acid+50 uL of dantrolene sodium)

70:30 (140 uL of dantrolene acid+60 uL of dantrolene sodium)

The effective pH of each sample was measured by making 10F dilutions in water. The pH values were recorded as follows—

90:10 pH 5.58

80:20 pH 672

75:25 pH 7.34

70:30 pH 9.00

100- and 200-fold dilutions of each of the four samples were made using 25% acetonitrile before being injected on the UPLC for analysis as described below. The dantrolene concentration of each sample was ˜9-10 mg/mL.

### Example 4. Lyophilization of Dantrolene Sodium

43.5 mgs of dantrolene sodium was slurried in 43.5 mL of water in a conical tube. The pH of the initial of the slurry was 9.89.

The pH of the slurry was then adjusted to 7.4 by incremental addition of aliquots of 1M HCl and 1M NaOH solutions.

Once at pH 7.4, the conical tube was vortexed, and a pH reading was taken (repeated a total of five times with a pH calibration in the middle to ensure that the meter was taking reliable measurements). The pH values ranged from 7.2-7.6, which is close to physiologic pH.

The top of the conical tube was covered with Parafilm, and holes were poked in the Parafilm to prepare the sample for lyophilization. Liquid nitrogen was used to flash freeze the sample before putting it in the lyophilizer. The sample remained on the lyophilizer for a total of four days.

### Example 5. Reconstitution of Lyophilized Dantrolene Sodium in PEG400

0.9 mgs of the lyophilized dantrolene sodium powder from Example 4 was reconstituted in 112 uL of PEG400. The sample went into solution within a few minutes after vortexing on and off. The sample was spun for 5 minutes at 15,000 rpm at room temperature to separate the supernatant (dantrolene sodium in solution) from the pellet (insoluble material). A small pellet formed after spinning. The supernatant was transferred into a clean tube and diluted 80- and 160-fold in 25% acetonitrile for analysis by UPLC as described herein. The solubility of dantrolene acid in PEG400 was calculated as ˜5 mg/mL.

The pH of the supernatant was measured by making a 10-fold dilution of the supernatant in water. The pH was measured as 5.02. The 10-fold dilution precipitated out quickly.

### Example 6. Reconstitution of Lyophilized Dantrolene Sodium in Propylene Glycol

1.2 mgs of lyophilized dantrolene sodium powder from Example 4 was reconstituted in 150 uL of propylene glycol. The sample became cloudy within a few minutes after vortexing on and off. The sample was spun for 5 minutes at 15,000 rpm at room temperature to separate the supernatant (dantrolene sodium in solution) from the pellet (insoluble material). A large pellet formed after spinning. The supernatant was transferred into a clean tube and diluted 50- and 100-fold in 25% acetonitrile for analysis by UPLC as described below. The solubility of dantrolene acid in propylene glycol was calculated as ˜0.6 mg/mL.

The pH of the supernatant was measured by making a 10-fold dilution of the supernatant in water. The pH was measured as 6.63. The 10-fold dilution precipitated out slowly.

### Example 7. Reconstitution of Lyophilized Dantrolene Sodium in Propylene Glycol:PEG400 50:50 Mix

1.1 mgs of the lyophilized dantrolene sodium powder from Example 4 was reconstituted in 68.7 uL of propylene glycol and 68.7 uL of PEG400. The sample became cloudy within a few minutes after vortexing on and off. The sample was spun for 5 minutes at 15,000 rpm at room temperature to separate the supernatant (dantrolene sodium in solution) from the pellet (insoluble material). A large pellet formed after spinning. The supernatant was transferred into a clean tube and diluted 50- and 100-fold in 25% acetonitrile for analysis by UPLC as described below. The solubility of dantrolene acid in propylene glycol:PEG400 50:50 mix was calculated as ˜2 mg/mL.

The pH of the supernatant was measured by making a 10-fold dilution of the supernatant in water. The pH was measured as 5.04. The 10-fold dilution precipitated out quickly.

### Example 8. Dissolution of Dantrolene Sodium in Propylene Glycol

Dantrolene sodium (6.4 mg) was weighed into a glass vial and propylene glycol (0.128 mL) was added, using a positive displacement pipette to achieve a target concentration of 50 mg/mL of dantrolene sodium. The contents of the vials were mixed with a positive displacement pipette. Visual inspection confirmed that the solutions were clear. The sample was a stable solution for at about 2 days at room temperature, after which time, precipitate formed. The precipitate was 99.9% dantrolene, as determined using HPLC.

Addition of 50 μL of water resulted in precipitation.

### Example 9. Dissolution of Dantrolene Sodium in PEG 400

Dantrolene sodium (6.2 mg) was weighed into a glass vial and PEG400 (0.124 mL) was added, using a positive displacement pipette to achieve a target concentration of 50 mg/mL of dantrolene sodium. The contents of the vials were mixed with a positive displacement pipette. Visual inspection confirmed that solutions were clear. The sample was a stable solution for at about 2 days at room temperature, after which time, precipitate formed. The precipitate was 99.9% dantrolene, as determined using HPLC.

Addition of 50 μL of water resulted in precipitation.

### Example 10. Concentration Determination by UPLC

Analysis was performed using an Agilent UPLC System equipped with a diode array detector and an Agilent C18 Zorbax column. Samples were analyzed using a gradient method with mobile phase A containing 0.1% trifluoroacetic acid in water and mobile phase B containing 0.1% trifluoroacetic acid in acetonitrile. The column was equilibrated with 75% mobile phase A. The % of mobile phase A was decreased to 57% A over the first 4 minutes. The column was then washed with 30% A for 2 minutes, returned to 75% over 0.1 minutes, and re-equilibrated with 75% A over 3.9 minutes for a total run time of 10 minutes. A 10 uL sample was injected and the analytes were detected by UV at 385 nm. The dantrolene eluted at approximately 2.3 minutes. Peak areas were determined by manual integration.

A standard curve was prepared by dissolving 1.0 mg of dantrolene sodium in 1 mL of 25% acetonitrile. This stock solution was further diluted in 25% acetonitrile to prepare standards at concentrations of 100, 50, 25, 12.5, and 6.25 ug/mL. The linear standard curve of peak area versus concentration was used to determine the concentration of unknown samples. The curve concentration values were adjusted to 5.84, 11.68, 23.36, 46.72 and 93.45 ug/mL to account for the difference in molecular weight between dantrolene acid (314 g/mole) and dantrolene sodium (336 g/mole).

### Example 11. Effective pH Determination

Effective pH measurements were conducted at ambient room temperature, using a Thermo Scientific Orion Star A111 pH benchtop meter. The instrument was calibrated within one day of the effective pH measurement using phosphate buffers at pH 4, 7, and 10 (Hydrion Tri-Chek Buffer Capsule set).

Samples were prepared by diluting the non-aqueous/anhydrous sample 10 fold with water. The diluted sample was vortexed for about 10 to 15 seconds. Effective pH was measured using the pre-calibrated pH benchtop meter, directly after vortexing.

After each effective pH measurement, the pH bulb was rinsed with an appropriate rinse solution to remove the entire test sample from the bulb.

1. A non-aqueous or anhydrous pharmaceutical composition comprising dantrolene, or a pharmaceutically acceptable salt thereof, or a mixture thereof, and a pharmaceutically acceptable carrier comprising a C₁₋₆alkyl alcohol, a polyol, a polyether, or a mixture thereof.

2. The pharmaceutical composition of claim 1, that is a non-aqueous pharmaceutical composition.

3. The pharmaceutical composition of claim 1, that is an anhydrous pharmaceutical composition.

4. The pharmaceutical composition of claim 1, having an effective pH of 3 to 11.5, preferably 4 to 9 or 5 to 8, more preferably having an effective pH of 7.4.

5. The pharmaceutical composition of claim 1, comprising dantrolene.

6. The pharmaceutical composition of claim 1, comprising dantrolene sodium.

7. The pharmaceutical composition of claim 1, comprising dantrolene and dantrolene sodium.

8. The pharmaceutical composition of claim 1, wherein the carrier is suitable for intravenous administration to a human.

9. The pharmaceutical composition of claim 1, wherein the carrier comprises ethanol, an alkylene glycol, or a liquid polyalkylene glycol, or a mixture thereof.

10. The pharmaceutical composition of claim 1, wherein the carrier comprises ethanol, an alkylene glycol, or a capped liquid polyalkylene glycol, or a mixture thereof.

11. The pharmaceutical composition of claim 1, wherein the carrier comprises ethanol, propylene glycol, or a polyethylene glycol, or a mixture thereof.

12. The pharmaceutical composition of claim 1, wherein the carrier comprises ethanol, propylene glycol, or a capped polyethylene glycol, or a mixture thereof.

13. The pharmaceutical composition of claim 1, wherein the carrier comprises propylene glycol or a polyethylene glycol or a mixture thereof.

14. The pharmaceutical composition of claim 1, wherein the carrier comprises propylene glycol.

15. The pharmaceutical composition of claim 1, wherein the carrier comprises polyethylene glycol.

16. The pharmaceutical composition of claim 1, further comprising an additional pharmaceutically acceptable excipient.

17. A method of treating a disorder responsive to dantrolene in a subject comprising administering to the subject a therapeutically effective amount of a pharmaceutical composition of claim 1.

18. The method of claim 17, wherein the disorder is malignant hyperthermia, chronic spasticity, exertional heat stroke, cardiac arrhythmias, tachycardis, atrial fibrillation, cardiac arrest, myocardial infarction, heart failure, myocardial injury, cardiomyopathy, central core disease, amyotrophic lateral sclerosis, rhabdomyolysis, Duchenne muscular dystrophy, ataxia, detrusor overactivity, overactive bladder, seizure, epilepsy, neuroleptic malignant syndrome, human stress disorder, Alzheimer's disease, Huntington's disease, multiple sclerosis, Parkinson's disease, ischemia-reperfusion injury, neuronal reperfusion injury, hypoxia, cerebral aneurysm, subarachnoid hemorrhage, stroke, hyperthermia associated with drug abuse, hyperthermia associated with drug overdose, nerve agent exposure, or acetylcholine accumulation.

19. The method of claim 17, wherein the administration is intravenous administration.

20. The method of claim 17, wherein the administration is intramuscular administration or subcutaneous administration.

21. The method of claim 17, wherein the administration is oral administration or intranasal administration.

22. The method of claim 17, wherein the administration is intraosseous administration.

23. A lyophilized pharmaceutical composition comprising dantrolene and a pharmaceutically acceptable salt of dantrolene.

24. The lyophilized pharmaceutical composition of claim 23, exhibiting an effective pH of 4 to 9 when reconstituted with a pharmaceutically acceptable carrier that is a C₁₋₆alkyl alcohol, a polyol, a polyether, or a mixture thereof.

25. A method of treating a disorder responsive to dantrolene in a subject comprising
reconstituting a lyophilized pharmaceutical composition comprising dantrolene and a pharmaceutically acceptable salt of dantrolene with a pharmaceutically acceptable carrier that is a C₁₋₆ alkyl alcohol, a polyol, a polyether, or a mixture thereof, to produce a reconstituted solution comprising dantrolene and a pharmaceutically acceptable salt of dantrolene having an effective pH of 4 to 9;
diluting the reconstituted solution with an additional pharmaceutically acceptable carrier to produce a diluted reconstituted solution comprising dantrolene and a pharmaceutically acceptable salt of dantrolene; and
administering a therapeutically effective amount of the diluted reconstituted solution to the subject.

26. A method of treating a disorder responsive to dantrolene in a subject comprising
diluting a pharmaceutical composition of claim 1 with an additional pharmaceutically acceptable carrier to produce a diluted pharmaceutical composition comprising dantrolene and a pharmaceutically acceptable salt of dantrolene; and
administering a therapeutically effective amount of the diluted pharmaceutical composition to the subject.

27. The method of claim 25, wherein the disorder is malignant hyperthermia, chronic spasticity, exertional heat stroke, cardiac arrhythmias, tachycardis, atrial fibrillation, cardiac arrest, myocardial infarction, heart failure, myocardial injury, cardiomyopathy, central core disease, amyotrophic lateral sclerosis, rhabdomyolysis, Duchenne muscular dystrophy, ataxia, detrusor overactivity, overactive bladder, seizure, epilepsy, neuroleptic malignant syndrome, human stress disorder, Alzheimer's disease, Huntington's disease, multiple sclerosis, Parkinson's disease, ischemia-reperfusion injury, neuronal reperfusion injury, hypoxia, cerebral aneurysm, subarachnoid hemorrhage, stroke, hyperthermia associated with drug abuse, hyperthermia associated with drug overdose, nerve agent exposure, or acetylcholine accumulation.

28. The method of claim 25, wherein the administration is intravenous administration.

29. The method of claim 25, wherein the administration is intramuscular administration or subcutaneous administration.

30. The method of claim 25, wherein the administration is oral administration or intranasal administration.

31. The method of claim 25, wherein the administration is intraosseous administration.

32. The method of claim 25, wherein the pharmaceutically acceptable dantrolene salt is dantrolene sodium.
